(19) United States
(12) Patent Application Publication
(10) Pub. No.: US 2006/0064131 A1
(43) Pub. Date: Mar. 23, 2006

(54) User interface for defibrillator for use by persons with limited training and experience

(76) Inventors: Gary A. Freeman, Newton Center, MA (US); Ward Hamilton, Amherst, NH (US); Frederick W. Faller, Burlington, MA (US)

Correspondence Address: FISH & RICHARDSON PC, P.O. BOX 1022, MINNEAPOLIS, MN, 55440-1022, US

(21) Appl. No.: 11/226,491

(22) Filed: Sep. 13, 2005

Related U.S. Application Data
(63) Continuation-in-part of application No. 10/804,312, filed on Mar. 18, 2004.
Application No. 10/804,312 is a continuation of application No. 09/794,320, filed on Feb. 27, 2001, now abandoned.
Application No. 09/794,320 is a continuation-in-part of application No. 09/498,306, filed on Feb. 4, 2000, now abandoned.
Application No. 09/794,320 is a continuation-in-part of application No. PCT/US01/03781, filed on Feb. 5, 2001.
(63) Continuation-in-part of application No. 09/952,834, filed on Sep. 14, 2001.

(51) Int. Cl. A61N 1/39

(52) U.S. Cl. 607/5

(57) ABSTRACT

A defibrillation system, comprising at least one first high-voltage defibrillation electrode, at least one second high-voltage defibrillation electrode, a control system electrically connected to the first and second electrodes and configured to cause a defibrillation shock to be applied to a patient through the first and second electrodes, and the control system being configured to pause at least about a second between activation of the defibrillation system and application of a shock to the patient.

### CROSS-REFERENCE TO RELATED APPLICATIONS

This application is a continuation-in-part (and claims the benefit of priority under 35 USC 120) of U.S. application Ser. No. 10/804,312, filed Mar. 18, 2004, which is a continuation of U.S. application Ser. No. 09/794,320, filed Feb. 27, 2001, now abandoned, which is a continuation-in-part of and claims priority to U.S. application Ser. No. 09/498,306, filed on Feb. 4, 2000, now abandoned, and PCT Application Serial No. PCT/US01/03781, filed on Feb. 5, 2001. This application is also a continuation-in-part (and claims the benefit of priority under 35 USC 120) of U.S. application Ser. No. 09/952,834, filed Sep. 14, 2001. The disclosure of the prior applications is considered part of (and is incorporated by reference in) the disclosure of this application.

### BACKGROUND

This invention relates to defibrillators for use by persons with limited training and experience.

Defibrillation can be performed with the use of an automatic external defibrillator (AED). Most automatic external defibrillators are actually semi-automatic external defibrillators (SAED), which require a clinician to press a start button, after which the defibrillator analyzes the patient's condition and provides a shock to the patient if the electrical rhythm is shockable and waits for user intervention before any subsequent shock. Fully automatic external defibrillators, on the other hand, do not wait for user intervention before applying subsequent shocks. As used below, automatic external defibrillators (AED) include semi-automatic external defibrillators (SAED).

Both types of defibrillators typically provide an oral stand clear warning before the application of each shock, and then the clinician is expected to stand clear of the patient and may be required to press a button indicating that the clinician is standing clear of the patient. The controls for automatic external defibrillators are typically located on a resuscitation control box.

AEDs are used typically by trained providers such as physicians, nurses, fire department personnel, and police officers. There might be one or two people at a given facility that has an AED who have been designated for defibrillation resuscitation before an ambulance service arrives. The availability of on-site AEDs along with rescuers trained to operate them is important because if the patient experiences a delay of more than 4 minutes before receiving a defibrillation shock the patient's chance of survival can drop dramatically. Many large cities and rural areas have low survival rates for defibrillation because the ambulance response time is slow, although many suburbs have higher survival rates because of the faster ambulance response time due to lack of traffic and availability of hospitals and advanced life support.

Trained lay providers are a new group of AED operators, but they rarely have opportunities to defibrillate. For example, spouses of heart attack victims may become lay providers, but these lay providers can be easily intimidated by an AED during a medical emergency. Consequently, such lay providers can be reluctant to purchase AEDs, or might tend to wait for an ambulance to arrive rather than use an available AED, out of concern that the lay provider might do something wrong.

### SUMMARY

It has been shown that trained lay providers, as well as untrained users, are wary of using AEDs because they are seen as delivering medical therapy, and because they are afraid of harming the patient with the shock. This becomes even more important for an AED designed for home use or in public areas by people who have had no training in AED use and little or no training in CPR. In many cases they will just not be willing to use the device and actually deliver the shock to the patient if expected to do so by using a button on the box labeled “SHOCK”.

It has been recognized in numerous studies and in various public forums that the time delay in delivering defibrillation therapy to a victim of cardiac arrest is the primary factor determining outcome. It has also been shown that 80% of all cardiac arrests occur in the home. Thus, it has been recognized that widespread and effective promulgation of AEDs into homes—much like fire extinguishers—will be a key determinant in the improvement in cardiac arrest survival rates, which currently stand at a dismal 3-5%. While medical device manufacturers, physicians, paramedics and governmental agencies such as the FDA still view these devices as therapeutic instruments requiring such activities as regulation and medical control, it is essential for the success of the effort to promulgate AEDs in the home that the devices are not only easy to use, as has been recognized by many authorities, but also that medical devices at least appear, in every respect, to the operator that they are in fact not medical devices.

It has been recognized by researchers when examining the effects of a rescue on the operator that there are significant post-rescue effects on the rescuer even in the case where the victim is saved. Post-traumatic stress disorder (PTSD) is a not uncommon side effect on the rescuer (more so sometimes than the victim who is unconscious at the time of the arrest). Considering that only one in twenty victims currently survive, and assuming that this survival rate can be quadrupled with the widespread distribution of AEDs, there will still only be one in five cardiac arrest victims who survive. As a result, 80% of all rescuers will need to deal with the guilt and potential PTSD of an unsuccessful rescue attempt. It is thus preferable in this situation that the rescuer not feel at any conscious or subconscious level that the death of the arrest victim was their fault.

The invention provides ways to suppress perceptual causality in AED therapy delivery.

In general, the invention features a defibrillation system, comprising at least one first high-voltage defibrillation electrode, at least one second high-voltage defibrillation electrode, a control system electrically connected to the first and second electrodes and configured to cause a defibrillation shock to be applied to a patient through the first and second electrodes, and the control system being configured to pause at least about a second between activation of the defibrillation system and application of a shock to the patient.

Preferred implementations of the invention may incorporate one or more of the following. The control system may be configured to provide resuscitation prompts to a rescuer for non-defibrillation resuscitation of the patient. The resuscitation prompts may comprise CPR prompts.

In a second aspect, the invention features an external defibrillator, comprising defibrillation electrodes for placement on the exterior of a patient, circuitry electrically connected to the electrodes and configured to deliver a defibrillation shock to the patient through the electrodes, a user-operable button electrically connected to the circuitry for initiating delivery of the defibrillation shock to the patient, wherein the circuitry is configured to deliver the defibrillation shock after a delay of between 300 and 1000 milliseconds following the user pressing the button.

Preferred implementations of the invention may incorporate one or more of the following. The delay may be between 500 and 1000 milliseconds. The delay may be between 700 and 1000 milliseconds.

In a third aspect, the invention features an external defibrillator, comprising defibrillation electrodes for placement on the exterior of a patient, circuitry electrically connected to the electrodes and configured to deliver a defibrillation shock to the patient through the electrodes, a user-operable button electrically connected to the circuitry for initiating delivery of the defibrillation shock to the patient, wherein the circuitry is configured to deliver the defibrillation shock after a delay of greater than 1000 milliseconds following the user pressing the button.

Preferred implementations of the invention may incorporate one or more of the following. The delay may be less than 1500 milliseconds.

In a fourth aspect, the invention features an external defibrillator, comprising defibrillation electrodes for placement on the exterior of a patient, circuitry electrically connected to the electrodes and configured to deliver a defibrillation shock to the patient through the electrodes, a user-operable button electrically connected to the circuitry for initiating delivery of the defibrillation shock to the patient, wherein the button provides no indication to the user that pressing it will directly initiate delivery of the shock.

Preferred implementations of the invention may incorporated one or more of the following. The button or adjacent areas may provide an indication that the user is prepared for the defibrillator to determine whether a shock should be delivered. The button or adjacent areas may provide an indication of one or a combination of the following: “ready”, “ok”, or a check mark.

In a fifth aspect, the invention features an external defibrillator, comprising defibrillation electrodes for placement on the exterior of a patient, circuitry electrically connected to the electrodes and configured to deliver a defibrillation shock to the patient through the electrodes, a user-operable button electrically connected to the circuitry for initiating delivery of the defibrillation shock to the patient, wherein the circuitry is configured to provide a prompt to the user following pressing of the button, and the prompt is designed to reinforce that pressing the button has not directly initiated delivery of the shock.

Preferred implementations of the invention may incorporated one or more of the following. The prompt may be an auditory spoken prompt or a verbal prompt viewable on a visual display. The prompt may convey to the user that the defibrillator is checking the condition of the patient.

Other features and advantages of the invention will be found in the detailed description, drawings, and claims.

### DESCRIPTION OF DRAWINGS

FIG. 1 is a drawing of a defibrillation electrode pad according to the invention, positioned over the chest of a patient.

FIG. 2 is a view of the front display panel of a resuscitation control box according to the invention that houses electronic circuitry and provides audible and visual prompting.

FIG. 3 is a cross-sectional drawing of the defibrillation electrode pad of FIG. 1 taken along line 3-3.

FIG. 4 is a cross-sectional drawing of the defibrillation pad of FIG. 1 taken along line 4-4.

FIG. 5 is a circuit diagram illustrating the circuit interconnections between the defibrillation electrode pad of FIG. 1 and the resuscitation control box of FIG. 2.

FIGS. 6A and 6B are a flowchart illustrating the initial routine of a resuscitation system according to the invention.

FIGS. 7A, 7B, and 7C are a flowchart illustrating the “circulation help” routine of the resuscitation system.

FIG. 8 is a flowchart illustrating the “breathing help” routine of the resuscitation system.

FIGS. 9A and 9B are a flowchart illustrating the “airway help” routine of the resuscitation system.

FIG. 10 is a flowchart showing a relationship found in some implementations between the user's pressing a button and the system's delivering a defibrillation shock.

### DETAILED DESCRIPTION

There are a great many possible implementations of the invention, too many to describe herein. Some possible implementations that are presently preferred are described below. It cannot be emphasized too strongly, however, that these are descriptions of implementations of the invention, and not descriptions of the invention, which is not limited to the detailed implementations described in this section but is described in broader terms in the claims.

Some implementations may use AED designs similar to those described in pending U.S. application Ser. No. 10/314,667 (published as US 2003/0083699; incorporated herein by reference), with modifications consistent with the claims appended hereto.

Other implementations may include a defibrillation and CPR assembly that combines traditional AED (automatic external defibrillation) functions with CPR prompting, and thus transforms a defibrillator into a resuscitation device that combines prompts for clearing a patient's airway, breathing, chest compression, and defibrillation.

Thus, the combined defibrillation and CPR assembly combines all of these aspects of resuscitation into a single protocol.

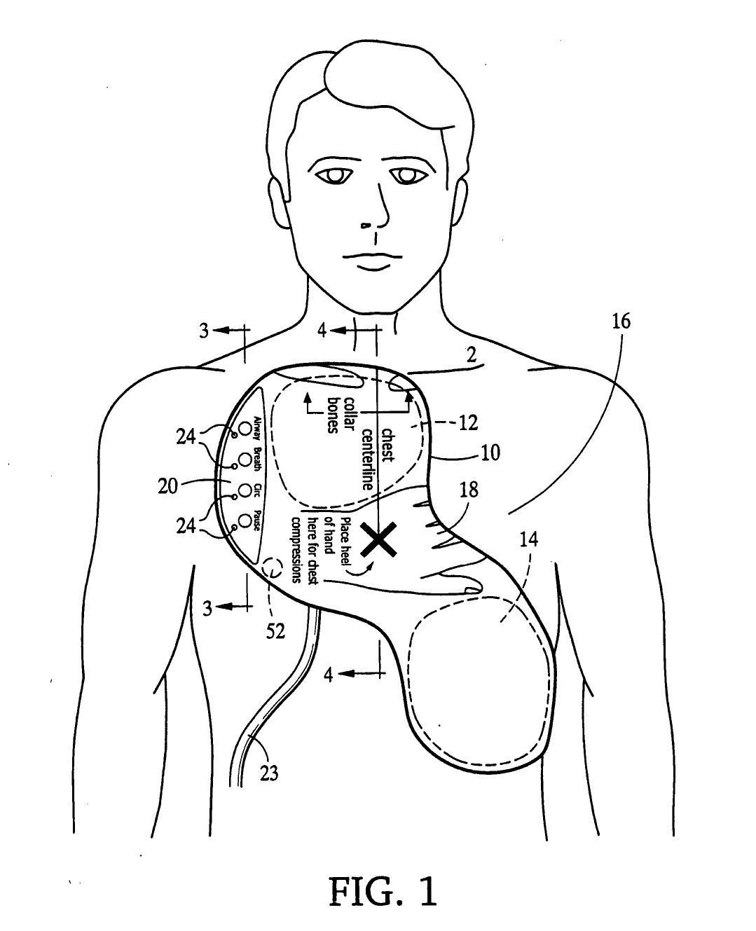

One possible implementation is shown in FIG. 1. A defibrillation electrode pad 10, which includes high-voltage apex defibrillation electrode 12 and high-voltage sternum defibrillation electrode 14, is placed on the patient's chest 16 and includes a region 18 on which a user may press to perform CPR. Legends on pad 10 indicate proper placement of the pad with respect to the patient's collarbones and the chest centerline and the proper placement of the heel of the rescuer's hand.

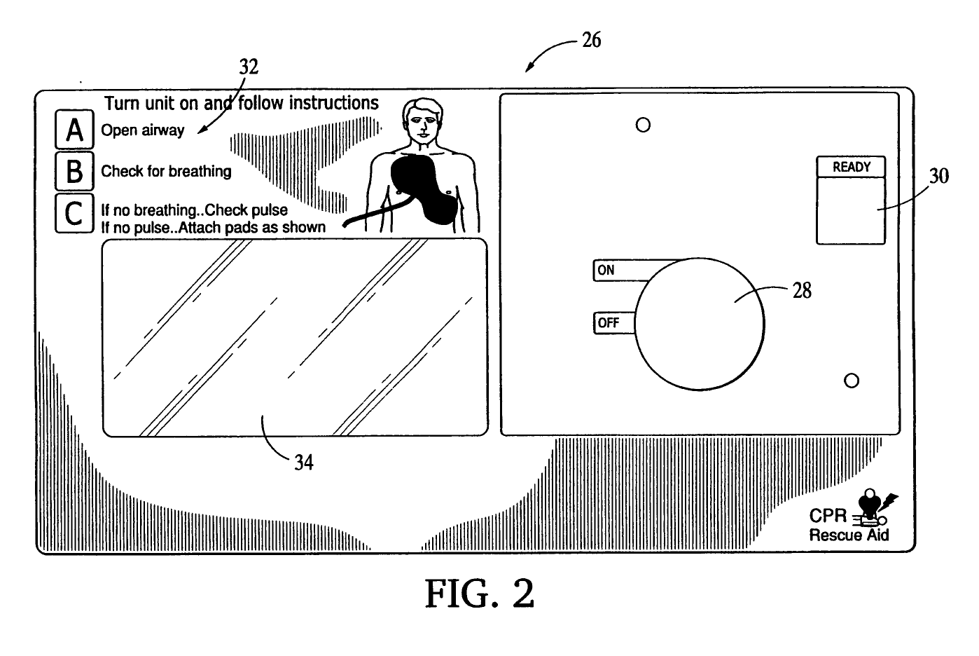

A low-profile button panel 20 is provided on the electrode assembly. Button panel 20 has buttons 22, including buttons A (Airway Help), B (Breathing Help), C (Circulation Help) and PAUSE, and may also include adjacent light emitting diodes (LEDs) 24 that indicate which button has been most recently pressed. Button panel 20 is connected by a cable 23 to a remote resuscitation control box 26, shown in FIG. 2. Button panel 20 provides rigid support underneath buttons A, B, C, and PAUSE against which the switches can be pushed in order to ensure good switch closure while the electrode rests on a patient. Button panel 20 includes components that make electrical contact with silver/silver-chloride electrical circuit components screen-printed on a polyester base of defibrillation electrode pad 10, as is described in detail below.

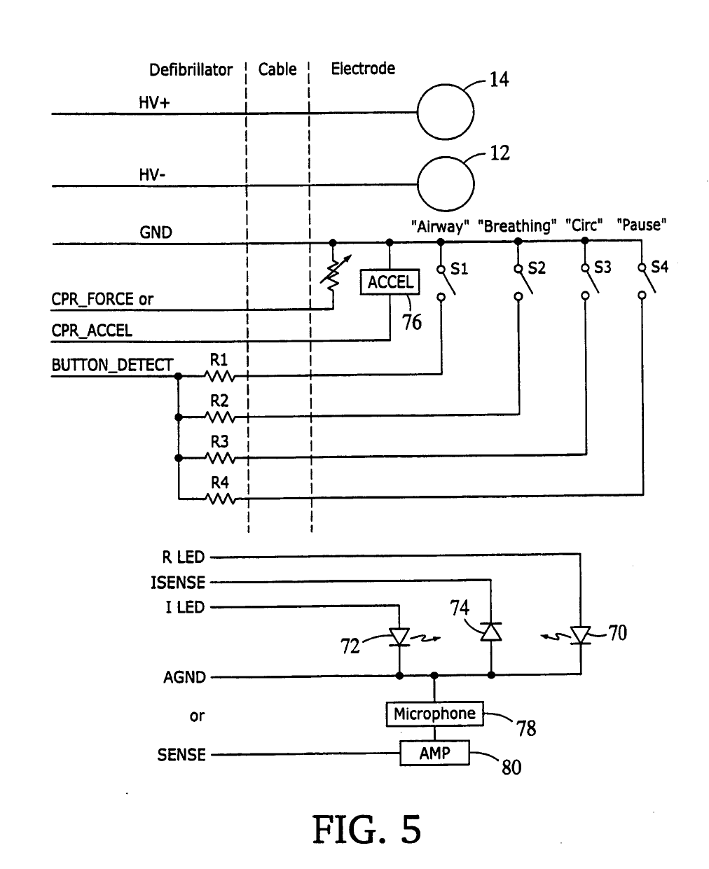

A pulse detection system based on shining light through the patient's vascular bed, e.g., a pulse oximetry system 52, is incorporated into defibrillation electrode pad 10. Pulse oximetry system 52 includes a red light-emitting diode, a near-infrared light-emitting diode, and a photodetector diode (see FIG. 5) incorporated into defibrillation electrode pad 10 in a manner so as to contact the surface of the patient's chest 16. The red and near-infrared light-emitting diodes emit light at two different wavelengths, which is diffusely scattered through the patient's tissue and detected by the photodetector diode. The information obtained from the photodetector diode can be used to determine whether the patient's blood is oxygenated, according to known noninvasive optical monitoring techniques.

In an alternative embodiment, the pulse detection system is a phonocardiogram system for listening to the sound of the victim's heart, rather than a pulse oximetry system. The phonocardiogram system includes a microphone and an amplifier incorporated within the electrode pad. Because a heart sound can be confused with microphone noise, the signal processing that must be performed by the microprocessor inside the control box will be more difficult in connection with a phonocardiogram system than in connection with a pulse oximetry system. Nevertheless, there are programs available that can enable the microprocessor to determine whether an ECG signal is present as opposed to microphone noise.

Pulse oximetry is a well-developed, established technology, but it requires good contact between the light sources and the victim's skin so that light can shine down into the victim's vascular bed. Many victims have lots of chest hair, which can interfere with good contact. It may be desirable for different types of electrode pads to be available at a given location (one having a pulse oximetry system and one having a phonocardiogram system) so that a rescuer can select an appropriate electrode pad depending on the nature of the victim.

In an alternative embodiment, instead of providing a low-profile button panel, a button housing can be provided that is affixed to an edge of the defibrillation electrode. The housing may be in the form of a clamshell formed of single molded plastic element having a hinge at an edge of the clamshell around which the plastic bends. The two halves of the clamshell can be snapped together around the electrode assembly.

The resuscitation control box (FIG. 2) includes an internal charge storage capacitor and associated circuitry including a microprocessor, an further includes off/on dial 28, and a “READY” button 30 that the rescuer presses immediately prior to application of a defibrillation shock in order to ensure that the rescuer is not in physical contact with the patient. The microprocessor may be a RISC processor such as a Hitachi SH-3, which can interface well with displays and keyboards, or more generally a processor capable of handling DSP-type (digital signal processing) operations.

The resuscitation control box has printed instructions 32 on its front face listing the basic steps A, B, and C for resuscitating a patient and giving basic instructions for positioning the defibrillation electrode pad on the patient. A speaker 32 orally prompts the user to perform various steps, as is described in detail below.

For example, the resuscitation control box instructs the user, by audible instructions and also through a display 34 on the resuscitation control box, to check the patient's airway and perform mouth-to-mouth resuscitation, and if the patient's airway is still blocked, to press the A (Airway Help) button on the button panel (FIG. 1), upon which the resuscitation control box gives detailed prompts for clearing the patient's airway. If the patient's airway is clear and the patient has a pulse but the patient does not breathe after initial mouth-to-mouth resuscitation, the resuscitation control box instructs the user press the B (Breathing Help) button, upon which the resuscitation control box gives detailed mouth-to-mouth resuscitation prompts. If, during the detailed mouth-to-mouth resuscitation procedure, the rescuer checks the patient's pulse and discovers that the patient has no pulse, the resuscitation control box instructs the user to press the C (Circulation Help) button.

During the circulation procedure, the resuscitation control box receives electrical signals from the defibrillation electrodes and determines whether defibrillation or CPR should be performed. If the resuscitation control box determines that defibrillation is desirable, the resuscitation control box instructs the user to press the “ready” button on the resuscitation control box and to stand clear of the patient. After a short pause, the resuscitation control box causes a defibrillation pulse to be applied between the electrodes. If at any point the resuscitation control box determines, based on the electrical signals received from the electrodes, that CPR is desirable, it will instruct the user to perform CPR.

In some implementations, the controls for the system may be on the electrodes attached to the patient rather than the resuscitation control box. This may be important because it enables the rescuer to remain focused on the patient rather than the control box. The resuscitation control box gets its information directly from the electrodes and the controls on the electrodes.

The resuscitation control box can sense electrical signals from the patient's body during pauses between CPR compressions. In other implementations, e.g., as described in U.S. application Ser. No. 10/844,253 (filed May 12, 2004), the patient's ECG may be sensed and processed during CPR compressions. Also, as is described below, a compression-sensing element such as an accelerometer or a force-sensing element is provided in the region of the defibrillation electrode pad on which the user presses to perform CPR. The purpose of the compression-sensing or force-sensing element is to allow the resuscitation control box to prompt the user to apply additional compression or force, or to prompt the user to cease CPR if the user is performing CPR at an inappropriate point in time.

Referring to FIG. 4, according to one embodiment of the invention, each electrode 12, 14 (only electrode 12 is shown) of defibrillation electrode pad 10 includes a polymer-based ink containing a silver/silver-chloride suspension, which is screen-printed on a polyester or plastic base 36. The ink is used to carry the defibrillation current. The screen-printing process first involves applying a resist layer to the polyester base 36. The resist layer is basically a loose mesh of nylon or the like, in which the holes have been filled in at some locations in the mesh. Then, the silver/silver-chloride ink is applied as a paste through the resist layer in a squeegee-like manner. The ink squeezes through the screen and becomes a solid layer. The ink may then be cured or dried. The silver/silver-chloride ink provides good conductivity and good monitoring capabilities.

Thus, the ink can be applied as pattern, as opposed to a solid sheet covering the entire polyester base. For example, U.S. Pat. No. 5,330,526 describes an electrode in which the conductive portion has a scalloped or daisy shape that increases the circumference of the conductive portion and reduces burning of the patient. A conductive adhesive gel 38 covers the exposed surface of each electrode.

In addition, electrical circuit components are also be screen printed on the base, in the same manner as flat circuit components of membrane-covered, laminated panel controls.

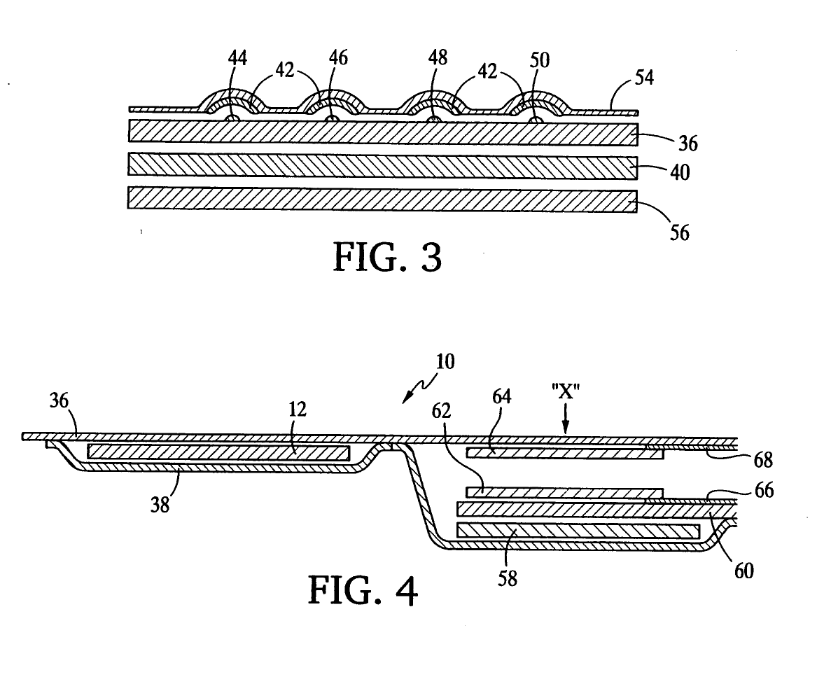

Referring to FIG. 3, a rigid piece 40 of hard plastic, such as PVC or polycarbonate, is laminated beneath substrate 36 and supports buttons A, B, C, and PAUSE. The rigid plastic piece 40 is glued onto substrate 36. Buttons A, B, C, and PAUSE consist of small metal dome snap-action switches that make contact between an upper conductive ink trace 42 and lower conductive ink traces 44, 46, 48, and 50. Buttons A, B, C, and PAUSE serve as controls that can be activated by the user that are physically located either on or immediately adjacent to the electrode assembly itself. Each of buttons A, B, C, and PAUSE may be associated with an adjacent light-emitting diode (LED). For example, LEDs may be glued, using conductive epoxy, onto silver/silver-chloride traces on substrate 36. An embossed polyester laminate layer 54 covers conductive ink trace 42 of buttons A, B, C, and PAUSE, and a foam layer 56 is laminated beneath rigid plastic piece 40.

Referring again to FIG. 4, defibrillation electrode pad 10 includes an extension piece that is placed directly over the location on the patient's body where the rescuer performs chest compressions. This extension piece includes substrate 36, and a semi-rigid plastic supporting member 58 laminated underneath substrate 36 that covers the chest compression area. Semi-rigid supporting member 58 provides somewhat less rigidity than rigid plastic piece 409 provided at the location of buttons A, B, C, and 30 PAUSE (illustrated in FIG. 3).

In embodiments having a force-sensing element, a polyester laminate 60, and a force-sensing resistor having two layers of carbon-plated material 62 and 64, are laminated between polyester substrate 36 and semi-rigid supporting member 58. A suitable construction of the force-sensing resistor is illustrated in the FSR Integration Guide & Evaluation Parts Catalog with Suggested Electrical Interfaces, from Interlink Electronics. The electrical contact between the two carbon-plated layers of material increases with increased pressure, and the layers of force-sensing resistive material can provide a generally linear relationship between resistance and force. Conductive ink traces 66 and 68 provide electrical connections to the two layers of the force-sensing resistor.

During chest compressions, the rescuer's hands are placed over the extension piece, and the force-sensing resistor of the extension piece is used to sense the force and the timing of the chest compressions. The force-sensing resistor provides information to the resuscitation control box so that the resuscitation control box can provide the rescuer with feedback if the rescuer is applying insufficient force. The resuscitation control box also provides coaching as to the rate at which CPR is performed. In certain situations, the resuscitation control box indicates to the rescuer that CPR should be halted because it is being performed at an inappropriate time, such as immediately prior to application of a defibrillation shock when the rescuer's hands should not be touching the patient, in which case the resuscitation control box will also indicate that the rescuer should stay clear of the patient because the patient is going to experience a defibrillation shock.

As is noted above, during CPR the rescuer pushes on the patient's chest through the extension piece in the vicinity of the electrodes. If the resuscitation control box were to perform analysis during the chest compressions, the chest compressions would be likely to affect the sensed electrical rhythm. Instead, during the pauses between sets of compressions (for example, the pause after every fifth chest compression), the resuscitation control box can perform an electrocardiogram (ECG) analysis. The resuscitation control box might discover, for example, that the patient who is undergoing CPR is experiencing a non-shockable rhythm such as bradycardia, in which case the CPR is required in order to keep the patient alive, but then the resuscitation control box may discover that the rhythm has changed to ventricular fibrillation in the midst of CPR, in which case the resuscitation control box would instruct the rescuer to stop performing CPR so as to allow the resuscitation control box to perform more analysis and possibly apply one or more shocks to the patient. Thus, the invention integrates the rescuer into a sophisticated scheme that allows complex combinations of therapy.

In an alternative embodiment, a compression-sensing element such as an accelerometer may be used in place of a force-sensing element. The accelerometer, such as a solid-state ADXL202 accelerometer, is positioned at the location where the rescuer performs chest compressions. In this embodiment, the microprocessor obtains acceleration readings from the accelerometer at fixed time intervals such as one-millisecond intervals, and the microprocessor integrates the acceleration readings to provide a measurement of chest compression. The use of an accelerometer is based on the discovery that it is more important to measure how deeply the rescuer is compressing the chest than to measure how hard the rescuer is pressing. In fact, every victim's chest will have a different compliance, and it is important that the chest be compressed about an inch and a half to two inches in a normal sized adult regardless of the victim's chest compliance.

FIG. 5 is a circuit diagram illustrating the circuit interconnections between the defibrillation electrode pad of FIG. 1 through the cable to the resuscitation control box of FIG. 2. Sternum electrode 14 is connected to HV+ at the resuscitation control box, and apex electrode 12 is connected to HV−. A ground GND is connected to the upper conductive ink trace of buttons A, B, C, and PAUSE and to one of the layers of the force-sensing resistor. The other layer of the force-sensing resistor is connected to CPR_FORCE, and the lower conductive ink traces associated with buttons A, B, C, and PAUSE are connected to BUTTON_DETECT through resistors R1, R2, R3, and R4. As an alternative to the use of a force-sensing resistor, a compression-sensing accelerometer 76 may be employed, in which case CPR_FORCE is replaced by CPR_ACCEL connected to accelerometer 76. Red light-emitting diode 70, near-infrared light-emitting diode 72, and photodetector diode 74 of the pulse oximetry system are connected to RLED, ILED, and ISENSE respectively, as well as ground AGND. As an alternative to the use of a pulse oximetry system, a phonocardiogram system may be employed, in which case RLED, ILED, and ISENSE is replaced by SENSE connected to microphone 78 and amplifier 80.

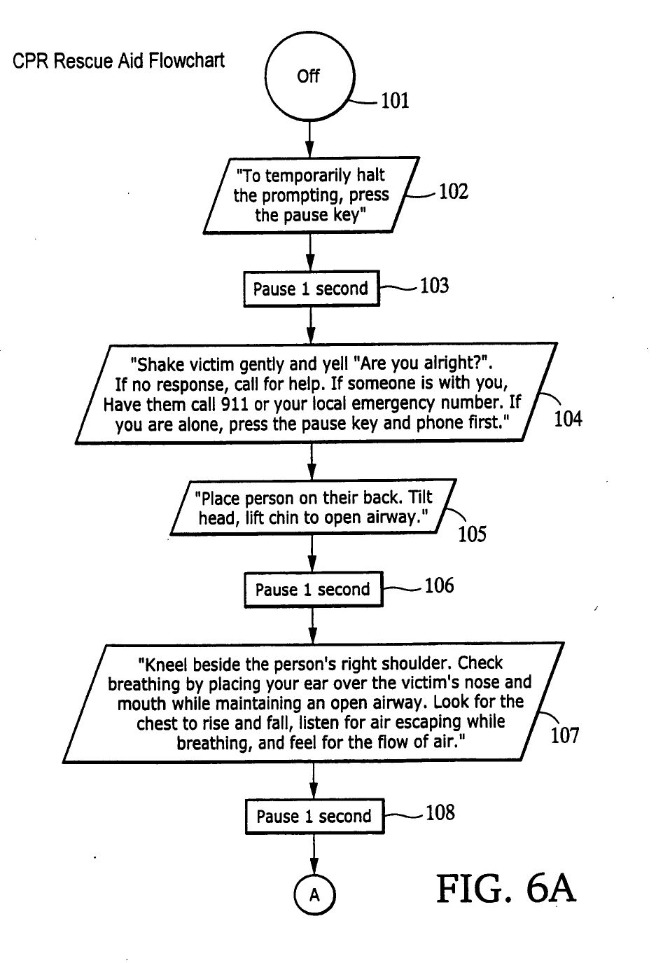

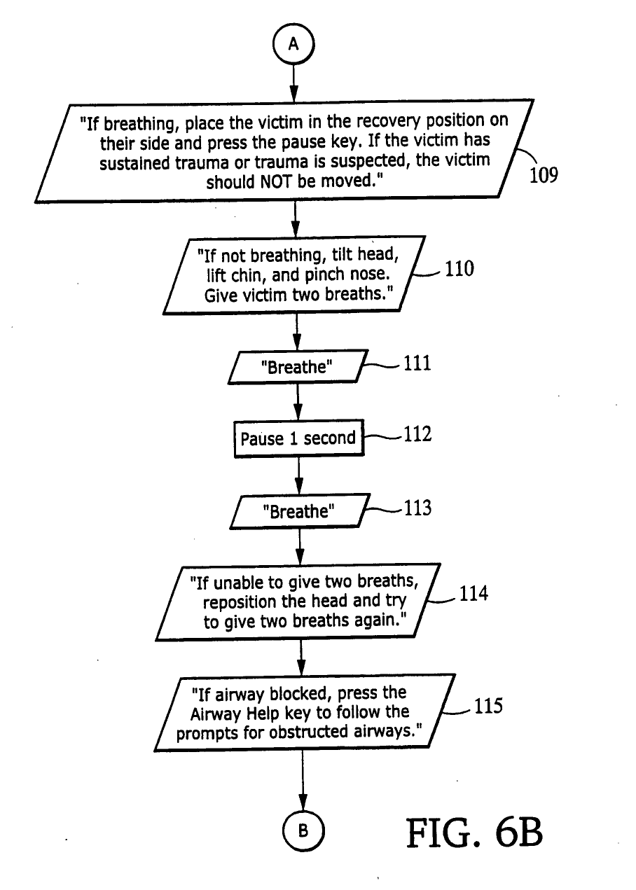

FIGS. 6-9 illustrate the routine of the resuscitation system described above, which is based on steps A, B, and C (airway, breathing, and circulation). Because step C includes defibrillation as well as chest compressions, all of the aspects of resuscitation are tied together in one protocol (actually, if defibrillation were considered to be a step D distinct from step C, the sequence of steps would be A, B, D, C).

The first thing the rescuer must do upon arriving at the patient is to determine whether the patient is unconscious and breathing. The rescuer opens the patient's airway, administers breaths to the patient if the patient is not breathing, and checks to determine whether a pulse is present. If there is no pulse, rather than perform chest compressions as in standard CPR, the rescuer allows the resuscitation control box to analyze the patient's electrical rhythm, and if the resuscitation control box determines that the rhythm is shockable, the resuscitation control box causes one or more shocks to be applied to the patient, and then the rescuer performs chest compressions. Thus, the invention provides a first response system that can keep the patient viable until an advanced life support time arrives to perform advanced techniques including pacing, further defibrillation, and drug therapy.

If the resuscitation control box determines that it should apply one or more defibrillation shocks to the patient, it is important that the rescuer not be anywhere near the patient when the shocks are applied to the patient. Prior to application of each shock, the resuscitation control box instructs the rescuer to please press the “ready” button when everybody is clear of the patient. The pressing of the “ready” button verifies that the rescuer's hands are off of the patient.

When the resuscitation control box detects a shockable rhythm, the resuscitation control box provides shocks of appropriate duration and energy (such as a sequence of shocks of increasing energy from 200 Joules to 300 Joules to the highest setting, 360 Joules, with the resuscitation control box performing analysis after each shock to determine whether another shock is required). If the defibrillation therapy is successful, the patient's rhythm is typically converted from ventricular fibrillation, ventricular tachycardia, or ventricular flutter to bradycardia, idio-ventricular rhythm, or asystole, all of which require CPR. It is rare to convert to a normal rhythm. Once the resuscitation control box has caused defibrillation shocks to be applied to the patient, the resuscitation control box automatically senses the patient's condition, and depending on the patient's condition will either prompt the responder to perform CPR or will not prompt the respond to perform CPR.

Defibrillation equipment can be somewhat intimidating to rescuers who are not medical professionals because the equipment can lead the rescuer to feel responsibility for having to save a loved one's life. It is important that the defibrillation equipment reduce this sense of responsibility. In particular, when the rescuer presses the “ready” button, rather than apply a shock immediately that will cause the patient's body to jump dramatically, the resuscitation control box will thank the rescuer and instruct the rescuer to remain clear of the patient and then wait for about two seconds (the resuscitation control box may describe this period to the rescuer as being an internal safety check, even if no substantial safety check is being performed). This process has an effect similar to a conversation that hands responsibility to the resuscitation control box, which makes the decision whether to apply the shock.

In some implementations, the system maintains the rescuer safety features of a semi-automatic external defibrillator, because the rescuer must press the “ready” button before each shock, while appearing to operate more as a fully automatic external defibrillator because the time delay immediately prior to each shock leaves the rescuer with the impression that operation of the equipment is out of the hands of the rescuer. The use of CPR prompts in combination with the defibrillation also adds to the sense that the rescuer is simply following instructions from the resuscitation control box.

In some implementations, in which an AED is designed to be used by average citizens such as the elderly spouse, wife, or child of the victim, in such environments as the home or public venues, no reference is provided on the key used to initiate the shock that the purpose of the key is to deliver either a shock or more generically to deliver a therapeutic treatment in any way. Instead, there are markings on the device or key itself that indicates that the operator should press that key at the appropriate time. A hand with a finger pushing on the key is one implementation. Another might be a label either on the device or key indicating that the operator was ready, e.g. “ready”. Another might be an iconic line drawing of a figure with their hands raised indicating that they are not touching the patient. In the medical, paramedic vernacular this is termed, “standing clear” of the patient or simply, “clear”. The key could also have the specific words, “clear”, that when pressed would indicate that the operator was clear of the patient and the device would then be able to safely deliver the defibrillation therapy.

In some implementations, there is also a short time delay that further separates the action of pressing a key from the event of defibrillation, thereby removing a causal link between the two, from the psychological perspective of the operator. In some implementations, a delay of 300 to 500 milliseconds is introduced subsequent to the operator pressing the “READY” key before a shock is delivered. In other implementations, the delay is 500 to 700 milliseconds. In still other implementations, the delay is 700 to 1000 milliseconds. And in other implementations the delay is greater than 1000 milliseconds.

The Belgian psychologist Albert Michotte developed theories on the perception of causality, positing that the perception of causality is a spontaneous perceptual gestalt, which is neither learned nor an interpretation via abstract knowledge of physical events. Michotte claimed that the essence of perceived causality is “ampliation of motion.” The neologism, “ampliation,” refers to two aspects of the perceived motion. In Michotte's case, he was describing the psychology of perception of a first ball striking a second ball. First, the motion of the approaching object is transferred to the launched object. Second, for a brief time just after impact (approximately 200 ms), the motion is phenomenologically duplicitous: it belongs to the first object while the second object has it. Thereafter, the motion of the second object becomes autonomous. While the term “Ampliation”, and the general concepts embodied in it, was first applied to the situation in which two objects such as billiard balls collide, it has come to be used more broadly in the psychology of perception of causality. Thus, if a second phenomenological event is perceived to be initiated within a perceptually causal interval (e.g. <300 milliseconds) of an instance of a first phenomenological event, and within the same perceptual frame as the first event, the second phenomenological event will be perceived as having been caused by the first.

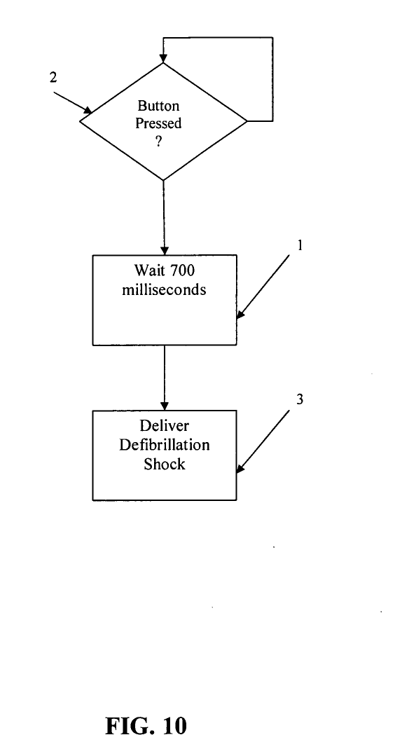

Referring to FIG. 10, in one implementation, the delay 1 between detection of button closure 2 and delivery of the defibrillation shock 3 is 700 milliseconds. If the delay is too long, e.g. greater than 2000 (or 1500) milliseconds, the operator may be unsure whether the device is functioning and may engage in needless activity. Additionally, the device may respond with a voice prompt of “ready”, after the “ready” button has been pressed, acknowledging the key press. The delay and/or the voice prompt “ready” may serve to suppress the rescuer's association of the pressing of the key and the delivery of therapy by the device.

The device may also include a voice prompt to the operator that tells the operator, “Make sure that no one is touching the patient and press the Ready key when Ready.”

The wording on the key may also be a colloquial word such as “OK” or simply the graphical checkmark ✓.

In an effort to increase the temporal isolation of the key press and the shock, pressing such a key may initiate a longer sequence of actions by the device such as ECG waveform analysis and automatically shocking if the waveform was found to be shockable. The device may provide an initial voice prompt such as, “Make sure that no one is touching the victim and the press the check key.” Subsequent to detection of the Check key press and the initiation of ECG analysis, the device would provide an acknowledging voice prompt indicating that the device was checking the patient's condition: “Beginning status check. Make sure not to touch the victim during this time.” After this prompt is completed and an intervening period of silence exceeds 1500 milliseconds, a short prompt, “Don't touch patient”, is delivered to make sure the rescuer understands that a continuing action by the device is in progress and no other action than staying clear of the patient is required of them. Specific wording may be chosen that is both non-medical and non-inciting. By such a method, the operator may be presented with a perceptual sequence that provides significant temporal isolation of his/her actions and the delivery of therapy to the patient, and with the general perception that it is the device that is the responsible party in treating the victim.

In some implementations (FIGS. 6-9), when the rescuer turns the resuscitation control box on (step 101), the resuscitation control box first informs the rescuer that the rescuer can temporarily halt prompting by pressing the PAUSE button (step 102), and then, after a pause, instructs the rescuer to check responsiveness of patient, and if the patient is non-responsive to call an emergency medical service (EMS) (steps 103, 104). The resuscitation control box then instructs the rescuer to check the patient's airway to determine whether the patient is breathing (steps 105-107).

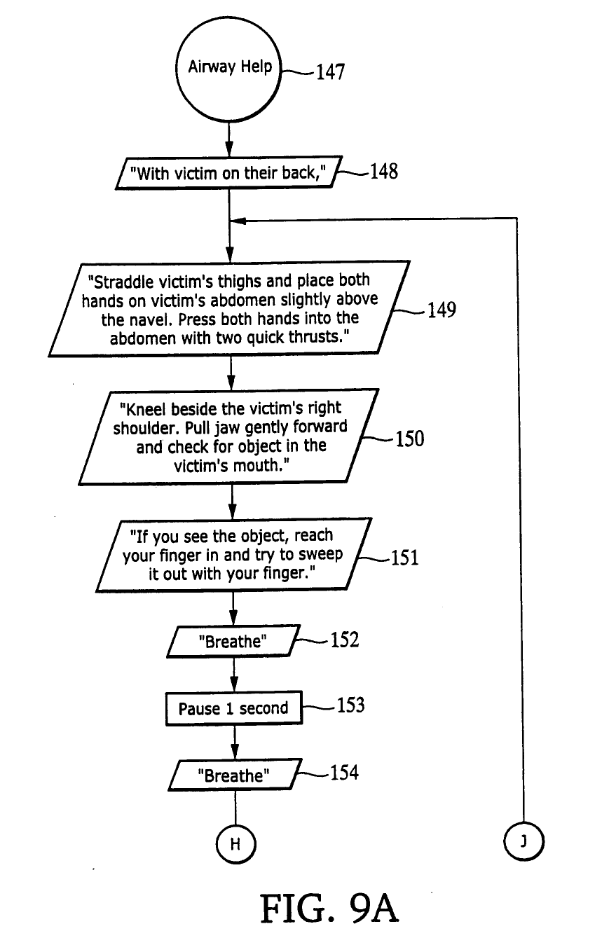

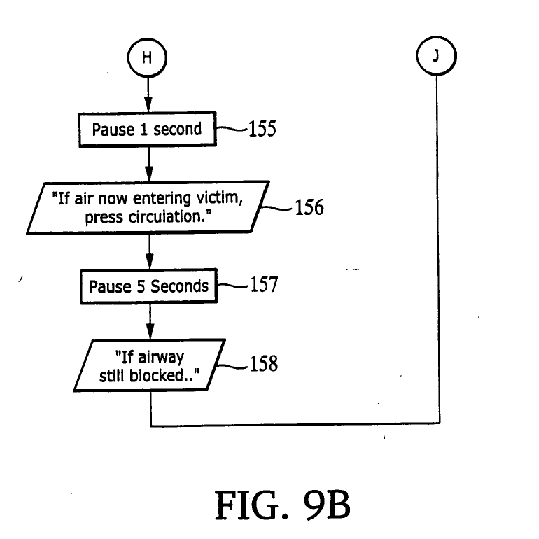

After a pause, the resuscitation control box then instructs the rescuer that if the patient is breathing the patient should be placed on the patient's side, unless trauma is suspected, and that the rescuer should press the PAUSE button (steps 108-109). Then the resuscitation control box instructs the rescuer to perform mouth-to-mouth resuscitation if the patient is not breathing (steps 110-114). Then the resuscitation control box instructs the rescuer to press an Airway Help button A if the patient's airway is blocked, so that the resuscitation control box can give prompts for clearing obstructed airways (steps 115 of FIG. 6B and 147-158 of FIGS. 9A-9B).

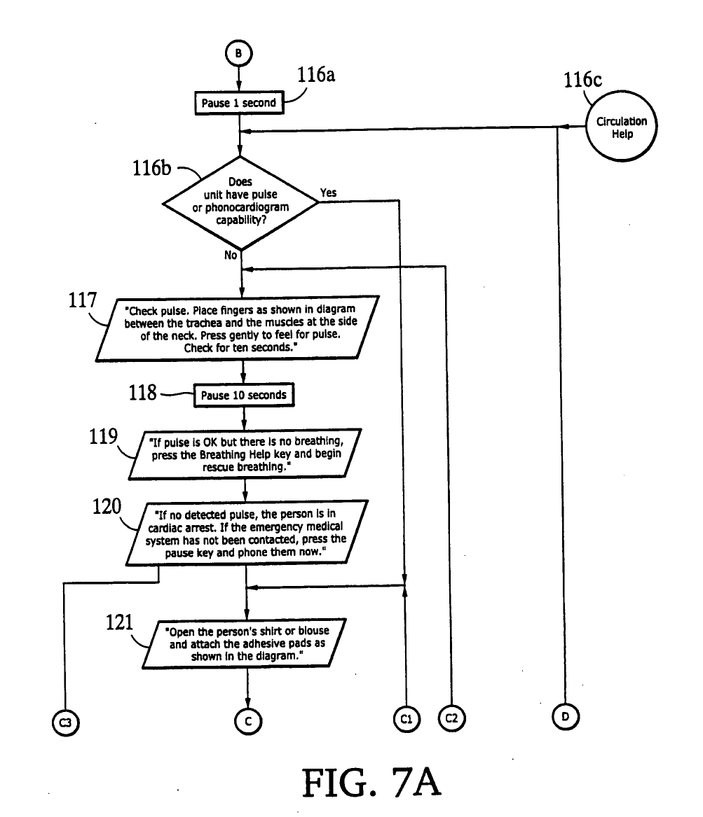

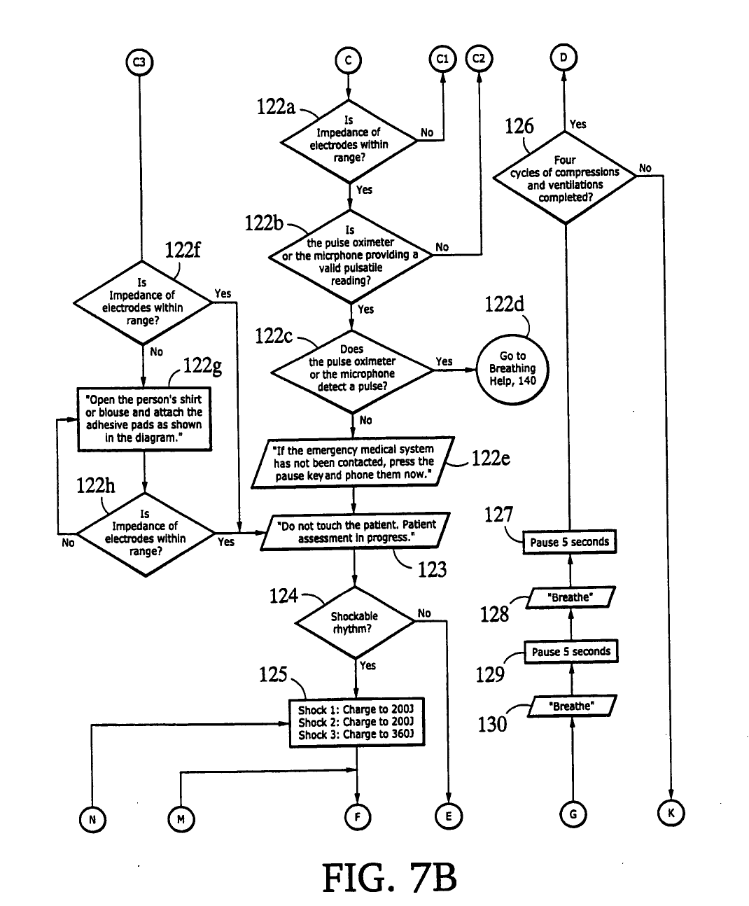

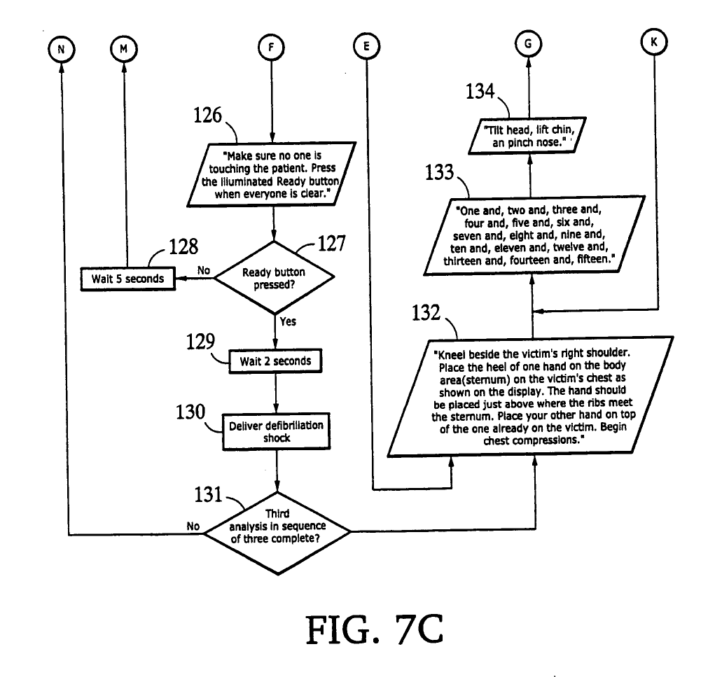

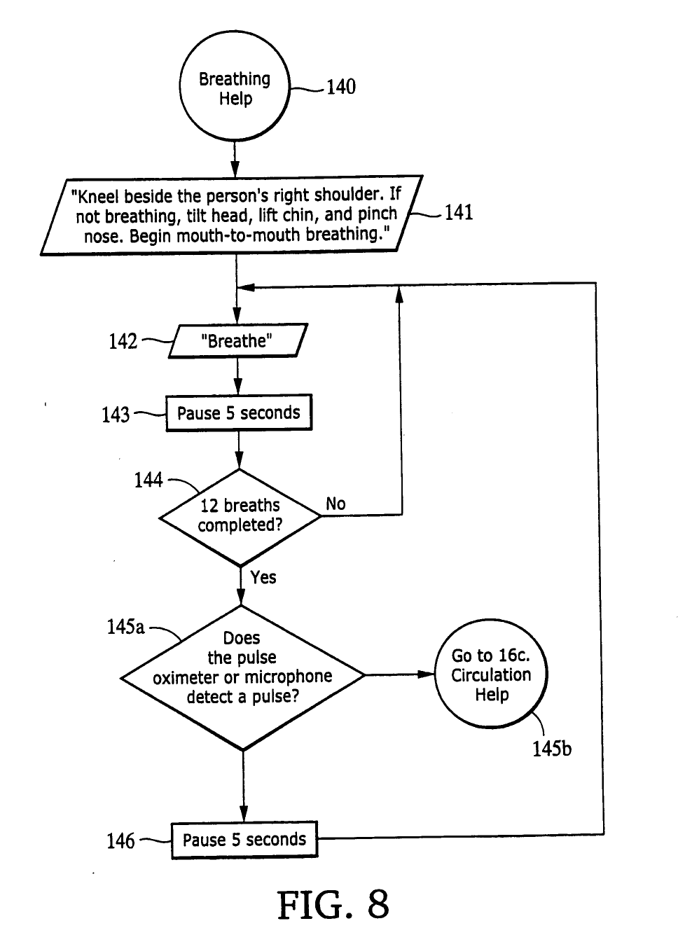

Next, after a pause (step 116a), if the resuscitation control box does not include pulse oximetry or phonocardiogram capability (step 116b), the resuscitation control box instructs the rescuer to check the patient's pulse (step 117). After another pause, the resuscitation control box instructs the rescuer to press a Breathing Help button B if the patient's pulse is okay but the patient is not breathing, so that the resuscitation control box can give prompts for assisting the patient's breathing (steps 118 and 119 of FIG. 7A and 140-146 of FIG. 8). Light-emitting diodes adjacent the various buttons indicate which button has been pressed most recently (only one light remains on at a time). The resuscitation control box next prompts the rescuer to contact an emergency medical system (step 120) and to open the patient's shirt or blouse and attach the adhesive pads (steps 122f-122h).

If the resuscitation control box does include pulse oximetry or phonocardiogram capability (step and 116b), the resuscitation control box prompts the rescuer to open the patient's shirt or blouse and attach the adhesive pads (steps 121 and 122a). If the pulse oximetry or phonocardiogram system does not provide a valid pulsatile reading (step 122b), then the flow chart proceeds to step 117. If the pulse oximetry or phonocardiogram system does provide a valid pulsatile reading and detects a pulse (steps 122b and 122c), then the resuscitation control box begins the breathing help routine (steps 122d of FIG. 7B and step 140 of FIG. 8). If the pulse oximetry or phonocardiogram system does not detect a pulse, then the resuscitation control prompts the rescuer to contact an emergency medical system (step 122e), measures the impedance of the patient to determine whether it is within an acceptable range for application of shocks (step 123) and determines whether the patient's rhythm is shockable (steps 124). If the rhythm is shockable, the resuscitation control box causes a sequence of shocks to be applied to the patient, each shock requiring the rescuer first to press the “READY” button on the resuscitation control box (steps 124-131). After the last shock in the sequence, or if the rhythm is non-shockable, the resuscitation control box prompts the rescuer in CPR (steps 132-139). The flowchart then returns to step 117.

FIG. 8 shows the steps 140-146 for prompting the rescuer to assist the patient's breathing. After 12 breaths have been completed (step 144), the pulse oximetry or phonocardiogram system attempts to detect a pulse (step 145a), or, if the system does not include a pulse oximetry or phonocardiogram system, the resuscitation control box prompts the rescuer to check the patient's pulse. If no pulse is present, the resuscitation control box prompts the rescuer to press a Circulation Help button C (step 145b) that brings the rescuer back to the circulation portion of the flowchart. Otherwise, if a pulse is detected, then the flow chart of FIG. 8 returns to step 142.

The combined defibrillation and CPR resuscitation assembly provided by the invention can be less intimidating than conventional AEDs because the assembly is not devoted solely to defibrillation. Moreover, the resuscitation assembly is less intimidating because it accommodates common skill retention problems with respect to necessary techniques ancillary to defibrillation such as mouth-to-mouth resuscitation and CPR, including the appropriate rates of chest compression, the proper location for performing compressions, the proper manner of tilting the patient's head. In addition, because the rescuer knows that it may never even be necessary to apply a defibrillation shock during use of the resuscitation assembly, the rescuer may be more comfortable using the resuscitation assembly for mouth-to-mouth resuscitation and CPR. Unlike previous CPR prompting devices, the rescuer would be required to place the electrode assembly on top of the patient, but the rescuer would do this with the belief that the resuscitation assembly will be sensing the patient's condition and that the likelihood that the resuscitation assembly is actually going to apply a shock is low. If, during this resuscitation process, the resuscitation control box instructs the rescuer to press the “READY” button so that a defibrillation shock can be applied, the rescuer will likely feel comfortable allowing the shock to be applied to the patient. Basically, the resuscitation assembly simply tells the rescuer what to do, and by that point, given that the rescuer is already using the assembly, the rescuer is likely simply to do what the rescuer is told to do. Essentially, the rescuer will be likely to view the resuscitation assembly as simply being a sophisticated CPR prompting device with an additional feature incorporated into it, and since rescuers are less likely to be intimidated by CPR prompting devices than AEDs, they will be likely to use the resuscitation assembly according to the invention when it is needed.

Other embodiments are within the following claims. For example, in other embodiments the system can perform pacing in addition to defibrillation.

1. A defibrillation system, comprising:
at least one first high-voltage defibrillation electrode;
at least one second high-voltage defibrillation electrode;
a control system electrically connected to the first and second electrodes and configured to cause a defibrillation shock to be applied to a patient through the first and second electrodes; and
the control system being configured to pause at least about a second between activation of the defibrillation system and application of a shock to the patient.

2. The defibrillation system of claim 1 wherein the control system is configured to provide resuscitation prompts to a rescuer for non-defibrillation resuscitation of the patient.

3. The defibrillation system of claim 2 wherein the resuscitation prompts comprises CPR prompts.

4. An external defibrillator, comprising:
defibrillation electrodes for placement on the exterior of a patient;
circuitry electrically connected to the electrodes and configured to deliver a defibrillation shock to the patient through the electrodes;
a user-operable button electrically connected to the circuitry for initiating delivery of the defibrillation shock to the patient, wherein the circuitry is configured to deliver the defibrillation shock after a delay of between 300 and 1000 milliseconds following the user pressing the button.

5. The defibrillator of claim 4 wherein the delay is between 500 and 1000 milliseconds.

6. The defibrillator of claim 5 wherein the delay is between 700 and 1000 milliseconds.

7. An external defibrillator, comprising:
defibrillation electrodes for placement on the exterior of a patient;
circuitry electrically connected to the electrodes and configured to deliver a defibrillation shock to the patient through the electrodes;
a user-operable button electrically connected to the circuitry for initiating delivery of the defibrillation shock to the patient,
wherein the circuitry is configured to deliver the defibrillation shock after a delay of greater than 1000 milliseconds following the user pressing the button.

8. The defibrillator of claim 7 wherein the delay is less than 1500 milliseconds.

9. An external defibrillator, comprising:
defibrillation electrodes for placement on the exterior of a patient;
circuitry electrically connected to the electrodes and configured to deliver a defibrillation shock to the patient through the electrodes;
a user-operable button electrically connected to the circuitry for initiating delivery of the defibrillation shock to the patient,
wherein the button provides no indication to the user that pressing it will directly initiate delivery of the shock.

10. The defibrillator of claim 9 wherein the button or adjacent areas provide an indication that the user is prepared for the defibrillator to determine whether a shock should be delivered.

11. The defibrillator of claim 10 wherein the button or adjacent areas provide an indication of one or a combination of the following: “ready”, “ok”, or a check mark.

12. An external defibrillator, comprising:
defibrillation electrodes for placement on the exterior of a patient;
circuitry electrically connected to the electrodes and configured to deliver a defibrillation shock to the patient through the electrodes;
a user-operable button electrically connected to the circuitry for initiating delivery of the defibrillation shock to the patient,
wherein the circuitry is configured to provide a prompt to the user following pressing of the button, and the prompt is designed to reinforce that pressing the button has not directly initiated delivery of the shock.

13. The defibrillator of claim 12 wherein the prompt is an auditory spoken prompt or a verbal prompt viewable on a visual display.

14. The defibrillator of claim 12 wherein the prompt conveys to the user that the defibrillator is checking the condition of the patient.
